(19) United States
(12) Patent Application Publication
(10) Pub. No.: US 2008/0220103 A1
(43) Pub. Date: Sep. 11, 2008

(54) METHOD FOR TREATING/CONTROLLING/KILLING FUNGI AND BACTERIA ON LIVING ANIMALS

(76) Inventors: Jay Birnbaum, Montville, NJ (US); Thomas Blake, Budd Lake, NJ (US); Mahmoud Ghannoum, Hudson, OH (US); Steven Vallespir, Park Ridge, NJ (US)

Correspondence Address: SONNENSCHEIN NATH & ROSENTHAL LLP, P.O. BOX 061080, WACKER DRIVE STATION, SEARS TOWER, CHICAGO, IL, 60606-1080, US

(21) Appl. No.: 12/047,992

(22) Filed: Mar. 13, 2008

Related U.S. Application Data
(63) Continuation-in-part of application No. 11/541,822, filed on Oct. 2, 2006.
(60) Provisional application No. 60/729,624, filed on Oct. 24, 2005.

(51) Int. Cl. A61K 36/00; A61K 36/899; A61K 36/534; A61K 36/61; A61K 36/736; A61K 31/045; A61K 31/05; A61K 31/35; A61K 31/11; A61K 31/192; A61K 31/4174; A61K 31/135; A61K 31/165; A61K 31/4412; A61P 31/10

(52) U.S. Cl. 424/735; 424/725; 424/747; 514/739; 514/731; 514/729; 514/456; 514/699; 514/570; 514/701; 514/399; 514/655; 514/599; 514/345

(57) ABSTRACT

Provided is a method of treating a fungal infection on an animal epidermis, nail or hair, or in an orifice of an animal. The method comprises contacting the fungus infection with a composition comprising an antifungal botanical.

### CROSS-REFERENCE TO RELATED APPLICATIONS

This application is a continuation-in-part of U.S. patent application Ser. No. 11/541,822, filed Oct. 2, 2006, which claims the benefit of U.S. Provisional Application No. 60/729,624 filed Oct. 24, 2005, both of which are incorporated herein by reference in their entirety.

### BACKGROUND OF THE INVENTION

(1) Field of the Invention

The present teachings relate to methods for treating/preventing animal diseases and odors associated with fungi and bacteria on surfaces or orifices of animals, including the skin, hair, nails, mouth, nose and vagina of the animal.

(2) Description of the Related Art

Fungal diseases are some of the most common affecting mammals, and include some of the most common infections in man. In humans these include, but are not limited to:

Tinea corporis—(“ringworm of the body”). This infection causes small, red spots that grow into large rings almost anywhere on the arms, legs, chest, or back.

Tinea pedis—fungal infection of the feet. Typically, the skin between the toes (interdigital tinea pedis or “Athlete's foot”) or on the bottom and sides of the foot (plantar or “moccasin type” tinea pedis) may be involved. Other areas of the foot may be involved.

Onychomycosis—fungal infection of the nail. The most prevalent type is the DSO or Distal Subungual Onychomycosis. Other types are White Superficial Onychomycosis, Proximal Subungual Onychomycosis, Candidal Onychomycosis, and Total Dystrophic Onychomycosis. These can be caused by various fungi (esp. dermatophytes=tinea unguium) and yeast, including Candida albicans.

Dandruff, which is the excessive shedding (exfoliation) of the epidermis of the scalp. A fungus may cause, or aggravate, the condition.

Tinea cruris: When the fungus grows in the moist, warm area of the groin, the rash is called tinea cruris. The common name for this infection is “jock itch.”

Tinea capitis, often called “ringworm of the scalp”, where the hair and scalp is affected, causes itchy, red areas, usually on the head. The hair is often destroyed, leaving bald patches. This tinea infection is most common in children, although a carrier state has been reported in adults.

Vaginal yeast infections, often caused by an overgrowth of a fungus that is a normal vaginal inhabitant, usually Candida albicans and Candida glabrata.

The list above providing but a few of the most common of a long list of such diseases in one mammal. Many diseases caused by fungi have been identified, and also include such common disease as oral thrush and diaper rash, often caused by members of the Candida genus. Fungi are often a complicating factor in diabetic and obese patients. In addition, disease in humans is caused by other fungi including but not limited to those from the genus Aspergillus, Blastomyces, Coccidioides, Cryptococcus, Histoplasma, Paracoccidioides, Sporothrix, and at least three genera of Zygomycetes, as well as those mentioned below under animals.

Secondary infections that can worsen diaper rash include fungal organisms (for example yeasts of the genus Candida).

The above fungi, as well as many other fungi, can cause disease in pets and companion animals. The present teaching is inclusive of substrates that contact animals directly or indirectly. Examples of organisms that cause disease in animals include Malassezia furfur, Epidermophyton floccosur, Trichophyton mentagrophytes, Trichophyton rubrum, Trichophyton tonsurans, Trichophyton equinum, Dermatophilus congolensis, Microsporum canis, Microsporu audouinii, Microsporum gypseum, Malassezia ovale, Pseudallescheria, Scopulariopsis, Scedosporium, and Candida albicans.

### BRIEF SUMMARY OF THE INVENTION

The inventors have discovered that some botanicals, singly or combined with other antifungal agents, are effective treatments for fungal diseases on surfaces or orifices of animals, including the skin, hair, nails, mouth, nose and vagina of the animal. The invention is thus directed to a method of treating a fungal infection on an animal epidermis, nail or hair, or in an orifice of an animal. The method comprises contacting the fungus infection with a composition comprising an antifungal botanical.

### BRIEF DESCRIPTION OF THE SEVERAL VIEWS OF THE DRAWINGS

FIG. 1 is photographs of Petri plates showing assays for antifungal activity. Panels A and B show a pre-treatment assay. In Panel A, active agents showed a clearance zone (arrow) around the biopsy disc, while (Panel B) inactive agents showed fungal growth around the disc. Panels C and D show a post-treatment assay. In Panel C, discs treated with active agents showed no fungal growth. In Panel D inactive agents showed fungal growth on discs.

FIG. 2 is photographs of Petri plates showing fungal growth assays using (A) CVS Double Air Foam Insoles, (B) Odor eater insoles, (C) CVS Odor Stop Insoles, (D) Dr Scholl's Air Pillow Insoles, (E) Control. None of these commercial insoles inhibited fungal growth.

FIG. 3 is photographs of Petri plates showing the effect of 30% isopropanol on Trichophyton mentagrophytes growth on (A) leather and (B) Dr. Scholl's insole. Isopropanol did not inhibit fungal growth.

FIG. 4 is graphs showing the effect of pretreatment of insoles (A) or leather (B) biopsy discs with different agents on growth of dermatophytes. Zone diameter indicates zone of clearance.

FIG. 5 is photographs of Petri plates showing the effect of pretreatment of insoles with (A) 1% terbinafine, (B) 1% tolnaftate, or (C) 1% tea tree oil.

FIG. 6 is photographs of Petri plates showing the effect of acetone on the activity of tolnaftate against dermatophyte growth. Panels A and B shows the growth of T. mentagrophytes on insole disc pretreated with (A) acetone or (B) 4% tolnaftate (w/v, prepared in acetone). Panel C shows the activity of 4% tolnaftate (dissolved in acetone) on already established contamination of T. mentagrophytes. (no fungal regrowth was observed).

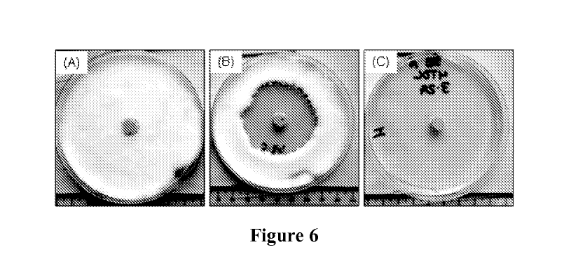

FIG. 7 is graphs showing the effect of post-infection treatment of insole (A) or leather (B) biopsy discs with different agents on dermatophyte growth. Zone diameter indicates zone of growth. Treatment with 30% isopropanol served as vehicle control.

FIG. 8 shows scanning electron microscopy (SEM) images of insoles infected with T. mentagrophytes. Magnification 2000× for all panels. Bar represents 20 μM for panels A through F, while it represents 100 M for the post-infected treated discs (Panels G-I).

FIG. 9 shows scanning electron microscopy (SEM) images of leather biopsies infected with T mentagrophytes. Magnification 2000×; bar −20 μm.

FIG. 10 is photographs of Petri plates showing assays for antifungal activity. The top panels (FIG. 10A) shows the effect of pretreatment of insoles with (A) 0.01% tolnaftate, (B) 3% tea tree oil, or (C) 0.01% tolnaftate+3% tea tree oil, on T. rubrum growth on shoe insoles. The lower panels (FIG. 10B) shows the effect of post-treatment of infected insoles with (A) 0.01% tolnaftate, (B) 3% tea tree oil, or (C) 0.01% tolnaftate+3% tea tree oil on T. rubrum growth.

### DETAILED DESCRIPTION OF THE INVENTION

### Abbreviations and Definitions

As used herein, a BOTANICAL is a compound isolated from a plant. Botanical antifungal compounds can be isolated from, for example, Ocimum basilicum (Basil), Cinnamomum aromaticum var. Cassia (Cinnamon), Cedrus libani (Cedar of Lebanon), any Cedrus spp., Chamaemelum nobile (Chamomile), Cymbopogon nardus (Citronella), Syzygium aromaticu (Clove & clove bud), Cuminum cyminum (Cumin), Foeniculum vulgare (Fennel), Melaleuca alternfolia (Tea Tree), Mentha x piperita (Peppermint), Mentha spicata (Spearmint), Curcuma longa (Tumeric), Cymbopogon citratus (Lemongrass), Santalum album (Sandalwood), as well as other compounds or ingredients, such as, but not limited to, eugenol, isolated from plants that have antifungal and/or antibacterial properties.

As used herein, a NATURAL ANTIFUNGAL COMPOUND (or naturally occurring antifungal compound) is a compound isolated from a botanical source (see botanical antifungal compound) or other naturally occurring source [e.g. mammalian cells, including but not limited to, neutrophils, or body fluid, e.g., saliva, amphibian skin, invertebrates (e.g. worms)]. These compounds can be proteins (e.g., enzymes), polysaccharides, small organic molecules, or other products produced by animals or plants.

A FUNGUS is any of numerous eukaryotic organisms of the kingdom Fungi (mycota), which lack chlorophyll and vascular tissue and range in form from a single cell (e.g., yeast) to a body of mass branched filamentous hyphae that often produce specialized fruiting bodies and pseudohyphae. The kingdom includes, but is not limited to, the yeasts, filamentous molds, dermatophytes, smuts, and mushrooms.

As used herein, an ANTIFUNGAL COMPOUND is defined herein as any chemical or substance that has the ability to inhibit the growth of fungi, and/or kill fungal cells or spores. Compound as used throughout this application includes salts and pro-drugs of the compound. Included in the definition of antifungal compound is any substance that possess static (e.g. inhibitory) activity (FUNGISTATIC COMPOUNDS) and/or cidal (e.g. killing) activity (FUNGICIDAL COMPOUNDS) against fungal cells (vegetative and spore structures). Also included in the definition of antifungal compound is any substance that is synthetic, semisynthetic or natural in origin that possesses antifungal activity as defined. “Antifungal compound” also includes any substance that can destroy/kill/inhibit the growth of fungal spores, for example, any substance that possesses a sporistatic (inhibitory) or sporicidal (killing) activity. See definition of Sporicidal compound below. Thus, throughout this document the term Antifungal compound is an all encompassing term referring to any substance (synthetic, semisynthetic, salt, pro-drug, natural, etc.) with antifungal activity, including, inhibitory, killing, static, cidal, sporistatic or sporicidal activity. These compounds can in turn be mixed with, for example, other antifungal compounds, detergents, and/or inactive ingredients to create formulation/s.

As used herein, a SPORE is a fungus in its dormant, protected state. It has a small, usually single-celled reproductive body that is highly resistant to desiccation and heat and is capable of growing into a new organism (vegetative state), produced especially by certain bacteria, fungi, algae, and non-flowering plants.

As used herein, a SPORICIDAL COMPOUND is a substance that either inhibits the growth of, increase the susceptibility of and/or destroys fungal spores. These can be synthetic, semisynthetic, or naturally occurring. Activating spores allows fungicides that only kill or inhibit actively growing fungi to kill those spores activated. This can be used, for example, in a mixture wherein a chemical(s) that activates growth is mixed with a chemical fungicide(s). It is also possible to use at least an activating compound alone, followed by at least a fungicide, serially. Activating spores is a method known in the art for bacterial spores, for example in U.S. Pat. No. 6,656,919, which is herein incorporated by reference.

As used herein, a BACTERICIDAL COMPOUND is a substance that either inhibits the growth of, increases the susceptibility of and/or destroys bacteria or bacteria spores. These can be synthetic, semisynthetic, or naturally occurring. Activating spores allows bactericides that only kill or inhibit actively growing bacteria to kill those spores activated. This can be used, for example, in a mixture wherein a chemical (s) that activates growth is mixed with a chemical bactericide(s). It is also possible to use at least an activating compound alone, followed by at least a bactericide, serially. Activating spores is a method known in the art for bacterial spores, for example in U.S. Pat. No. 6,656,919, which is herein incorporated by reference.

As used herein, an EPIDERMIS is the outer, protective, nonvascular layer of the skin of vertebrates, covering the dermis, it serves as the major barrier function of skin and is devoted to production of a cornified layer of the skin. Epidermally derived structures include hair (and fur), claws, nails, and hooves.

Treating an animal epidermis, nail, hair or orifice, means to contact, expose or apply a substance to the epidermis, nail, hair or orifice. This can include, but is not limited to, the delivery methods discussed below. A cream, ointment, gel, liquid, solution, foam, powder, paste, gum, lacquer, shampoo, suspension, fog, spray, aerosol, pump spray, wipe or sponge, or any other formulation, can include, for example, at least one fungicide. An effective treatment leads to the reduction in the presence of the infecting organism, an inhibition in growth of the infecting organism, and/or a reduction in the signs and/or symptoms of the infection, and need not necessarily lead to eradication of the infection.

MINIMAL INHIBITORY CONCENTRATION (MIC) is described, for instance, in Clin Infect Dis. 1997 February; 24(2):235-47. Tests for antifungal activity include MIC and MFC (Minimum Fungicidal Concentration) assays. These assays are used to determine the smallest amount of drug or compound needed to inhibit (MIC) or kill (MFC) the fungus.

Examples of antifungal compounds can be selected from the following chemical classes, or chemicals below, or naturally occurring compounds: aliphatic nitrogen compounds, amide compounds, acylamino acid compounds, allylamine compounds, anilide compounds, benzanilide compounds, benzylamine compounds, furanilide compounds, sulfonanilide compounds, benzamide compounds, furamide compounds, phenylsulfamide compounds, sulfonamide compounds, valinamide compounds, antibiotic compounds, strobilurin compounds, aromatic compounds, benzimidazole compounds, benzimidazole precursor compounds, benzothiazole compounds, bridged diphenyl compounds, carbamate compounds, benzimidazolylcarbamate compounds, carbanilate compounds, conazole compounds, conazole compounds (imidazoles), conazole compounds (triazoles), copper compounds, dicarboximide compounds, dichlorophenyl dicarboximide compounds, phthalimide compounds, dinitrophenol compounds, dithiocarbamate compounds, cyclic dithiocarbamate compounds, polymeric dithiocarbamate compounds, imidazole compounds, inorganic compounds, mercury compounds, inorganic mercury compounds, organomercury compounds, morpholine compounds, organophosphorus compounds, organotin compounds, oxathiin compounds, oxazole compounds, polyene compounds, polysulfide compounds, pyrazole compounds, pyridine compounds, pyrimidine compounds, pyrrole compounds, quinoline compounds, quinone compounds, quinoxaline compounds, thiocarbamate compounds, thiazole compounds, thiophene compounds, triazine compounds, triazole compounds, and urea compounds.

Antifungal compounds include the specific compounds albaconazole, amorolfine (dimethylmorpholine), amphotericin B, including lipid formulations of amphotericin B such as AmBisome and Abelcet, anidulafungin bifonazole, butenafine, butoconazole, caspofungin, clioquinol, ciclopirox olamine, clotrimazole, econazole, fluconazole, griseofulvin, haloprogen, iodochlorhydroxyquine, itraconazole, ketoconazole, miconazole, naftifine, oxiconazole, povidone-iodine sertaconazole, sulconazole, terbinafine, terconazole, tioconazole, tolnaftate, undecylenic acid and its salts (calcium, copper, and zinc), voriconazole, the sodium or zinc salts of proprionic acid, butylamine, cymoxanil, dodicin, dodine, guazatine, iminoctadine, carpropamid, chloraniformethan, cyflufenamid, diclocymet, ethaboxam, fenoxanil, flumetover, furametpyr, mandipropamid, penthiopyrad, prochloraz, quinazamid, silthiofam, triforine, benalaxyl, benalaxyl-M, furalaxyl, metalaxyl, metalaxyl-M, micafungin, pefurazoate, benalaxyl, benalaxyl-M, boscalid, carboxin, fenhexamid, metalaxyl, metalaxyl-M, metsulfovax, ofurace, oxadixyl, oxycarboxin, pyracarbolid, posaconazole, AN2690 (Anacor Pharmaceuticals), AN2718 (Anacor), thifluzamide, tiadinil, benodanil, flutolanil, mebenil, mepronil, salicylanilide, tecloftalam, fenfuram, furalaxyl, furcarbanil, methfuroxam, flusulfamide, benzohydroxamic acid, fluopicolide, tioxymid, trichlamide, zarilamid, zoxamide, cyclafuramid, furmecyclox, dichlofluanid, tolylfluanid, amisulbrom, cyazofamid, benthiavalicarb, iprovalicarb, aureofungin, blasticidin-S, cycloheximide, griseofulvin, kasugamycin, natamycin, polyoxins, polyoxorim, streptomycin, validamycin, azoxystrobin, dimoxystrobin, fluoxastrobin, kresoxim-methyl, metominostrobin, orysastrobin, picoxystrobin, pyraclostrobin, trifloxystrobin, biphenyl, chlorodinitronaphthalene, chloroneb, chlorothalonil, cresol, dicloran, hexachlorobenzene, pentachlorophenol, quintozene, sodium pentachlorophenoxide, tecnazene, benomyl, carbendazim, chlorfenazole, cypendazole, debacarb, fuberidazole, mecarbinzid, rabenzazole, thiabendazole, furophanate, thiophanate, thiophanate-methyl, bentaluron, chlobenthiazone, TCMTB, bithionol, dichlorophen, diphenylamine, benthiavalicarb, furophanate, iprovalicarb, propamocarb, thiophanate, thiophanate-methyl, benomyl, carbendazim, cypendazole, debacarb, mecarbinzid, diethofencarb, climbazole, imazalil, oxpoconazole, prochloraz, triflumizole, imidazole compounds, azaconazole, bromuconazole, cyproconazole, diclobutrazol, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, penconazole, propiconazole, prothioconazole, quinconazole, simeconazole, tebuconazole, tetraconazole, triadimefon, triadimenol, triticonazole, uniconazole, uniconazole-P, triazole compounds, Bordeaux mixture, Burgundy mixture, Cheshunt mixture, copper acetate, copper carbonate, basic, copper hydroxide, copper naphthenate, copper oleate, copper oxychloride, copper sulfate, copper sulfate, basic, copper zinc chromate, cufraneb, cuprobam, cuprous oxide, mancopper, oxine copper, famoxadone, fluoroimide, chlozolinate, dichlozoline, iprodione, isovaledione, myclozolin, procymidone, vinclozolin, captafol, captan, ditalimfos, folpet, thiochlorfenphim, binapacryl, dinobuton, dinocap, dinocap-4, dinocap-6, dinocton, dinopenton, dinosulfon, dinoterbon, DNOC, azithiram, carbamorph, cufraneb, cuprobam, disulfiram, ferbam, metam, nabam, tecoram, thiram, ziram, dazomet, etem, milneb, mancopper, mancozeb, maneb, metiram, polycarbamate, propineb, zineb, cyazofamid, fenamidone, fenapanil, glyodin, iprodione, isovaledione, pefurazoate, triazoxide, conazole compounds (imidazoles), potassium azide, potassium thiocyanate, sodium azide, sulfur, copper compounds, inorganic mercury compounds, mercuric chloride, mercuric oxide, mercurous chloride, (3-ethoxypropyl)mercury bromide, ethylmercury acetate, ethylmercury bromide, ethylmercury chloride, ethylmercury 2,3-dihydroxypropyl mercaptide, ethylmercury phosphate, N-(ethylmercury)-p-toluenesulphonanilide, hydrargaphen, 2-methoxyethylmercury chloride, methylmercury benzoate, methylmercury dicyandiamide, methylmercury pentachlorophenoxide, 8-phenylmercurioxyquinoline, phenylmercuriurea, phenylmercury acetate, phenylmercury chloride, phenylmercury derivative of pyrocatechol, phenylmercury nitrate, phenylmercury salicylate, thiomersal, tolylmercury acetate, aldimorph, benzamorf, carbamorph, dimethomorph, dodemorph, fenpropimorph, flumorph, tridemorph, ampropylfos, ditalimfos, edifenphos, fosetyl, hexylthiofos, iprobenfos, phosdiphen, pyrazophos, tolclofos-methyl, triamiphos, decafentin, fentin, tributyltin oxide, carboxin, oxycarboxin, chlozolinate, dichlozoline, drazoxolon, famoxadone, hymexazol, metazoxolon, myclozolin, oxadixyl, vinclozolin, barium polysulfide, calcium polysulfide, potassium polysulfide, sodium polysulfide, furametpyr, penthiopyrad, boscalid, buthiobate, dipyrithione, fluazinam, fluopicolide, pyridinitril, pyrifenox, pyroxychlor, pyroxyfur, bupirimate, cyprodinil, diflumetorim, dimethirimol, ethirimol, fenarimol, ferimzone, mepanipyrim, nuarimol, pyrimethanil, triarimol, fenpiclonil, fludioxonil, fluoroimide, ethoxyquin, halacrinate, 8-hydroxyquinoline sulfate, quinacetol, quinoxyfen, benquinox, chloranil, dichlone, dithianon, chinomethionat, chlorquinox, thioquinox, ethaboxam, etridiazole, metsulfovax, octhilinone, thiabendazole, thiadifluor, thifluzamide, methasulfocarb, prothiocarb, ethaboxam, silthiofam, anilazine, amisulbrom, bitertanol, fluotrimazole, triazbutil, conazole compounds (triazoles), bentaluron, pencycuron, quinazamid, acibenzolar, acypetacs, allyl alcohol, benzalkonium chloride, benzamacril, bethoxazin, carvone, chloropicrin, DBCP, dehydroacetic acid, diclomezine, diethyl pyrocarbonate, fenaminosulf, fenitropan, fenpropidin, formaldehyde, furfural, hexachlorobutadiene, iodomethane, isoprothiolane, methyl bromide, methyl isothiocyanate, metrafenone, nitrostyrene, nitrothal-isopropyl, OCH, 2-phenylphenol, phthalide, piperalin, probenazole, proquinazid, pyroquilon, sodium orthophenylphenoxide, spiroxamine, sultropen, thicyofen, tricyclazole, iodophor, silver, Nystatin, amphotericin B, griseofulvin, and zinc naphthenate.

The inventors have discovered that fungal infections on an animal epidermis, nail or hair, or in an orifice of an animal can be effectively treated with antifungal botanicals. Thus, provided herein are newly discovered properties of compounds which include antifungal, sporicidal and antibacterial properties. Also novel are the combinations of compounds which provide unexpected results in the treatment and pre-treatment of the epidermis, nail, hair and orifices against common fungi and bacteria. The inventors show for the first time that combining naturally occurring fungicides with known synthetic and semisynthetic fungicides leads to unexpectedly good results on substrates, including leather. The same results are now expected with living epidermis, nail or hair, or in an orifice. The Examples they also show that uses of naturally occurring fungicidal compounds can be expanded to be utilized against bacteria and resistant microorganisms. It is now concluded by the inventors that: 1) essential oils (especially lemongrass and clove bud oils, but not limited to them) can be used singly as natural products to inhibit microorganisms that infect epidermis, hair, nails, and orifices, and 2) combining essential oils with a synthetic or semisynthetic antifungal compound will provide a broad spectrum activity. In addition to treating common microorganisms, the methods of the invention can be employed to treat drug resistant microorganisms such as terbinafine resistant Trichophyton rubrum and multi-drug resistant Candida, as well as allow the use of lower concentrations of synthetic agents when combined with essential oils. The current method provides an effective means for preventing and treating fungal infection of the epidermis, hair, nails and orifices.

Having shown that antifungal compounds possess potent anti-dermatophyte activity, the inventors also showed the activity of these agents against dermatophytes, other fungi and bacteria using bioassays. See Examples.

The present invention is thus directed to a method of treating a fungal infection on an animal epidermis, nail or hair, or in an orifice of an animal. The method comprises contacting the fungus infection with a composition comprising an antifungal botanical.

In some embodiments, the fungal infection is in an orifice. As used herein, an orifice is an opening to a cavity or a passage of the body of a live animal (including a human). Examples of orifices include a mouth, nose, ear, vagina, anus, and urethra. Also included are artificially created orifices such as fistulas.

In some aspects of these embodiments, the orifice is a mouth or nose. In other aspects the orifice is a vagina.

In other embodiments, the fungal infection is on an epidermis.

In additional embodiments, the fungal infection is on a nail.

In some of the invention methods, the fungal infection causes or aggravates tinea corporis, tinea pedis, tinea cruris, tinea unguium, tinea capitis, dandruff, a vaginal yeast infection, or diaper rash.

In some embodiments, the fungal infection is of a Malassezia furfur, a Epidermophyton floccosum, a Trichophyton, a Dermatophilus congolensis, a Microsporum, a Malassezia ovale, an Aspergillus, a Blastomyces, a Candida, a Coccidioides, a Cryptococcus, a Histoplasma, a Paracoccidioides, a Sporothrix, a Zygomycetes, a Pseudallescheria, a Scedosporum, or a Scopulariopsis.

In some aspects of these methods, the antifungal botanical is an essential oil. Preferred essential oils are clove bud oil, lemongrass oil, sandalwood oil, spearmint oil, carcacrol, thymol, a cardamom extract, caraway oil, a coriander extract, linalool, almond oil, and tea tree oil. More preferably, the essential oil is clove bud oil or lemongrass oil. Most preferably, the essential oil comprises eugenol, terpinen-4-ol, cineole, cuminaldehyde, cinnamic acid, or perillaldehyde.

Combining agents can have a number of potential benefits, including: (a) extending and broadening the spectrum of activity of the individual agents used, (b) increase the antimicrobial potency of individual compounds, (c) reduce the development of resistance, (d) treat resistant strains (e) reduce the concentration used for at least one treatment agent, and/or (f) have anti-sporicidal activity with or without activating the spores. The data in the Examples herein shows that using a combination of the synthetic, semisynthetic, and natural products will achieve these objectives.

For example, since miconazole, unlike terbinafine and tolnaftate, possesses antibacterial activity, combining it with either agent will expand the spectrum of antimicrobial activity of an antifungal to cover dermatophytes, yeasts, and bacteria. In addition, because miconazole is a static agent against fungi, combining it with either terbinafine or tolnaftate, which we showed have fungal sporicidal activity, will expand the killing activity of the combination. These combinations are provided as examples and one of skill in the art can deduce from these teachings other effective combinations that can work synergistically.

The compositions used in the methods of the present invention can thus also comprise a second antifungal compound. In some embodiments, the second antifungal compound is a synthetic or a semisynthetic antifungal compound.

Where the second antifungal compound is a synthetic antifungal compound, preferred synthetic antifungal compounds are miconazole, terbinafine, tolnaftate or ciclopirox. Where the second antifungal compound is a semisynthetic antifungal compound, the preferred semisynthetic antifungal compound is an echinocandin.

In other embodiments, the second antifungal compound is a botanical. Preferably, the botanical is an essential oil, as described above.

The invention method can be used on any animal including a human, a nonhuman vertebrate such as a bird, or a non-human mammal. Examples of nonhuman vertebrates include any farm animal, for example a cow, a pig, a chicken, or a horse, or a companion animal such as a dog, a cat, a hamster, a gerbil, a guinea pig, a mouse, a rat, a potbellied pig, a ferret, or a caged bird.

The methods of the present invention are not narrowly limited to any particular method of contacting the compositions to the fungus infection. In some embodiments, the composition is applied directly on the fungal infected tissue. In other embodiments, the composition is applied to an article that comes in contact with the fungus infection. Preferred examples of such articles include, but are not limited to, clothing, a towel, a comb, a brush, a diaper, human bedding, or animal bedding.

The compositions used in the invention methods are not limited to any particular formulation, provided the formulation is pharmaceutically acceptable. By “pharmaceutically acceptable” it is meant a material that: (i) is compatible with the other ingredients of the composition without rendering the composition unsuitable for its intended purpose, and (ii) is suitable for use with subjects as provided herein without undue adverse side effects (such as toxicity, irritation, and allergic response). Side effects are “undue” when their risk outweighs the benefit provided by the composition. Non-limiting examples of pharmaceutically acceptable carriers include, without limitation, any of the standard pharmaceutical carriers such as phosphate buffered saline solutions, water, emulsions such as oil/water emulsions, microemulsions, nanoemulsions, and the like.

The use of particular excipients (detergents, oils, enzymes, etc.) can also function in the invention to increase the penetration of the substrate, the rate of penetration, the thoroughness of coverage, etc. These can also be used to cause the penetration of a spore or epidermis, hair or nails, or tissues of an orifice by an antifungal or antibacterial compound. Excipients can also be used to cause the spore to end dormancy and begin germination, thus making the spore more susceptible to the antifungal compound(s).

The composition comprising the antifungal or antibacterial compound can also include a compound to increase adherence to the epidermis, hair, nails or orifice. Increasing adherence can increase the length of time for which the compound remains in contact with the skin, hair and nails

The above-described compounds can thus be formulated without undue experimentation for administration to a mammal, including humans, as appropriate for the particular application. Additionally, proper dosages of the compositions can be determined without undue experimentation using standard dose-response protocols.

Non-limiting examples of forms of the compositions include a cream, ointment, gel, liquid, solution, foam, powder, paste, gum, lacquer, shampoo, suspension, fog, spray, aerosol, pump spray, wipe or sponge.

The methods of the present invention can further comprise contacting the fungal infection with a second composition. In these embodiments, the second composition comprises a separate antifungal compound. In some embodiments, the separate antifungal compound is a synthetic or semisynthetic antifungal compound. Where the separate antifungal compound is a synthetic antifungal compound, preferred separate antifungal compounds are miconazole, terbinafine, tolnaftate or ciclopirox. Where the separate antifungal compound is a semisynthetic antifungal compound, the preferred second antifungal compound is an echinocandin.

The separate antifungal compound can also be a botanical. Preferably, the botanical is an essential oil. Most preferably, the botanical comprises eugenol, terpinen-4-ol, cineole, cuminaldehyde, cinnamic acid, or perillaldehyde.

In addition, the compositions of the invention limit growth of odor causing bacteria, and the bacteria that cause cellulitis.

Preferred embodiments of the invention are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### EXAMPLES

### Example 1

### Examples of Antifungal Compounds that Function in the Invention

The treatment in this example consists of at least two antifungal compounds. Examples of typical compounds are listed by their general class, chemical or otherwise. Concentrations pertain to the class. They are stated as an overall range. One would select at least one compound from each group of antifungal compounds described above or below and create a mixture of the two or more compounds. All percents indicate weight/volume.

Imidazoles (0.01-10%): albaconazole, bifoconazole, butoconazole, clotrimazole, econazole, fluconazole, itraconazole, ketoconazole, miconazole, oxiconazole, posaconazole, saperconazole, AN 2690, sertaconazole, sulconazole, terconazole, tioconazole, voriconazole, luliconazole.

Allylamines And Benzylamines (0.001-10%; 0.05-5%): butenafine, naftifine, terbinafine.

Polyenes (0.01-10%; 0.5-5%): amphotericin B and its lipid preparations, candidicin, filipin, fungimycin, nystatin.

Miscellaneous Synthetic Antifungal Compounds (for example at 0.05-25%): amorolfine (demethymorpholine), cicloprox olamine, haloprogen, clioquinol, tolnaftate, undecylenic acid, hydantoin, chlordantoin, pyrroInitrin, salicylic acid, ticlatone, triacetin, griseofulvin, zinc pyrithione.

Disinfectants (for example at 0.001-20%): copper sulfate, Gentian Violet, betadyne/povidone iodine, colloidal silver, zinc.

Botanicals (for example at 0.01-10%): Basil (Oncimum basilicum), Cassia (Cinnamomum aromaticum var. cassia), Cedrus wood oil (Cedrus libani or Cedrus spp.), Chamomile (Chamaemelum nobile), Citronella (Cymbopogon nardus), Clove (Syzgium aromaticum), Cumin (Cuminum cyminum), Fennel (Foeniculum vulgare), Mint (Mentha x piperita/Mentha spicata), Tea Tree Oil (Melaleuca alternfolia), Tumeric leaf oil (Curcuma longa), Lemongrass Oil (Cymbopogon citratus), and ingredients isolated from these botanicals.

### Example 2

### Evaluation of the Activity of Synthetic Antifungal Compounds and Natural Substances Against Microorganisms Infecting Shoes Using In Vitro and Shoe and Insole Biopsy Disc Assays

The shoe disinfecting activities of the following compounds were studied: terbinafine, tolnaftate, miconazole, Cedrus oil, and tea tree oil, clove bud oil, lemongrass oil, sandalwood oil and spearmint oil.

### In Vitro Susceptibility Testing

Minimum Inhibitory Concentration (MIC): Minimum inhibitory concentrations of synthetic and semisynthetic and natural products against dermatophytes were determined using a modification of the Clinical Laboratory Standards Institute (CLSI, formerly National Committee of Clinical Laboratory Standards, NCCLS) M38A standard method for dermatophytes, while MIC of these agents against Candida species were determined using the CLSI M27-A2 methodology. The method used to determine the MIC against bacteria was based on the CLSI document M7-A7.

For dermatophytes, serial dilutions of terbinafine, tolnaftate were prepared in a range of 0.004-2 μg/ml, while for miconazole concentrations ranged between 0.015-8 μg/ml. Finally, for essential oils, the concentrations tested were between 0.03-16 μg/ml. The only exception was tea tree oil where dilutions were prepared in a range of 0.0078-4 μg/ml. The MIC was read at 4 days post inoculation and defined as the lowest concentration of an agent to inhibit 80% of fungal growth as compared to the growth control (Table 2).

To determine the MIC of agents against Candida species, serial dilutions of terbinafine and tolnaftate were prepared in a range of 0.125-64 μg/ml, miconazole in a range of 0.03-16 μg/ml and tea tree oil had a range between 0.125-4 μg/ml. The remaining essential oils were prepared in a dilution range of 0.03-16 μg/ml. For Candida the MIC was read at 24 hours and defined as the lowest concentration to inhibit 50% of fungal growth as compared to the growth control (Table 4).

For bacterial species, the medium used to evaluate the antibacterial activity of agents and essential oils was Mueller-Hinton (Oxoid Ltd., Basingstoke, Hampshire, England). Serial dilutions of miconazole, terbinafine, and tolnaftate were prepared in a range of 0.125-64 μg/ml and serial dilutions of tea tree oil were prepared in a range of 0.0078-4 μg/ml, while those for the rest of the essential oils were prepared in a range 0.031-16 μg/ml. The MIC was read at 16 h and defined as the lowest concentration to inhibit 80% of bacterial growth compared to the growth control (Table 5).

Minimum fungicidal concentration (MFC): The minimum fungicidal concentrations of various agents were determined using the technique described by Canton et al. (Antimicrob Agents Chemother. 2004 8:2477-82). In that method, fungal conidia were collected following growth on potato dextrose agar (PDA) plates and were used to inoculate 96-well plates containing different concentrations of agents. Following incubation at 35° C. for 4 days (for dermatophytes) or 24 hours (for yeast), wells showing no visible growth were cultured to determine the MFC (defined as the lowest concentration of a given agent that kills>99.999% of fungal conidia or spores). The MFC value represents the level of the agent at which spores or conidia were killed.

Evaluation of the activity of combination of antifungal agents and essential oils against microorganisms infecting substrates, including skin, hair, and nails.

Combining agents has a number of potential benefits, including: (a) extending and broadening the spectrum of activity of the individual agents used, (b) increase the antimicrobial effectiveness of individual compounds, (c) reduce the development of resistance, (d) treat resistant strains, (e) reduce the concentration used for at least one treatment agent, and (f) have sporicidal activity.

### Bioassay

The shoe substrate used in this study was Dr Scholl's<c air pillow insoles. This substrate was used in our bioassay because this insole has no inhibitory activity against dermatophytes (see below), and is representative of the type of material used in manufacturing shoe insoles.

To evaluate the ability of the agents to prevent and treat fungal contamination of insoles and leather, we determined their activity against the dermatophyte T. mentagrophytes, and developed a novel insole/leather biopsy assay. T. mentagrophytes was used as the model strain in our bioassay studies because this fungus is a major cause of tinea pedis and onychomycosis. Unlike T. rubrum, which is often identified as the causative organism in these diseases but is a poor producer of spores/conidia, T. mentagrophytes, in addition to being an etiological agent of these diseases, produces conidia reproducibly and therefore, is amenable for use in a bioassay. It is expected that activity in this assay against T. mentagrophytes will be indicative of activity against T. rubrum and other dermatophytes.

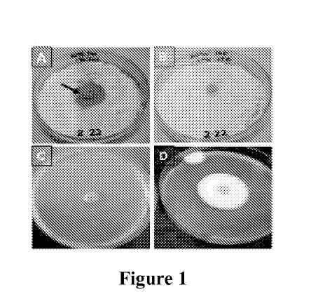

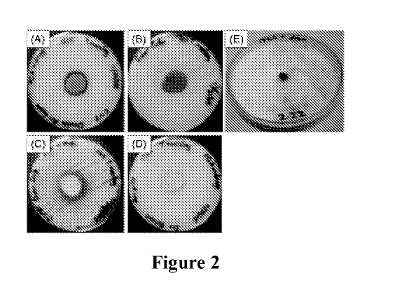

To evaluate the shoe disinfecting ability of various agents, a bioassay was developed that measured the activity of various agents in preventing (through pre-treatment) and treating (through post-treatment) contamination on shoes. The first step in the bioassay development was to identify optimal insole and leather material that represent substrates used in shoes and that do not inhibit fungi by themselves. To select the optimal shoe insole, discs measuring 8 mm were cut using a Dermal Biopsy punch (Miltex, Bethpage, N.Y.) from four commercially available shoe insoles (CVS odor stop insoles, Dr Scholl's air pillow insoles [which claim antifungal activity], odor eater insoles, and CVS double air foam insoles). These biopsy discs were placed on T. mentagrophytes seeded PDA plates. T. mentagrophytes was used as a typical organism and is representative of an entire class of fungi that grows on/in shoes and other substrates. The ability of insole biopsy discs from existing products to inhibit dermatophytes, following incubation for 7 days at 35° C., was determined (FIG. 1). Three of the insoles (CVS Odor Stop, Odor Eater, and CVS Double Air Foam) had a minimal antifungal activity (FIG. 2A-C) while Dr Scholl's insole did not inhibit T. mentagrophytes at all (FIG. 2D). A similar approach was used to determine whether biopsy discs from a leather hide inhibit fungal growth. As shown in FIG. 2E, the leather material did not have any antifungal activity by itself. Therefore, Dr Scholl's insole and the leather hide were used as substrates in subsequent experiments.

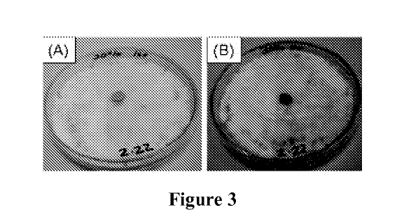

In our bioassay, we used isopropanol as a vehicle to dissolve the various disinfectants/antifungals. Isopropanol was selected because it is a common solvent used in different preparations marketed for the treatment of tinea pedis. We next performed experiments to identify a concentration of isopropanol that did not inhibit fungal growth by itself. The ability of three different concentrations (30%, 50%, and 100%) of isopropanol to inhibit dermatophyte growth was tested. As shown in FIGS. 3A-B, 30% isopropanol was the optimal concentration at which the vehicle did not inhibit fungal growth on the insoles and leather surface. Because tolnaftate does not dissolve very well in isopropanol, we performed additional experiments using acetone as a vehicle.

Based on the above experiments, our disc biopsy assay employed Dr. Scholl's insole and leather discs as the optimal substrates representing materials used in shoes, and 30% isopropanol as the optimal vehicle to dissolve the agents to be tested in pre-treatment and post-treatment studies.

### Evaluation of the Ability of Various Agents to Prevent and Treat Fungal Shoe Contamination.

Two types of disc biopsy assays were used to evaluate the ability of different synthetic and natural substances to disinfect shoe material: (a) Pre-treatment assay: where discs were pre-treated with antifungals first and then infected with T. mentagrophytes, and (b) Post-treatment assay: where discs were first infected with T. mentagrophytes, then treated with drugs. These assays reveal the ability of different agents to prevent and treat shoe fungal contamination, respectively.

Pre-treatment assays: To evaluate the ability of different agents to prevent fungal contaminations of shoes, PDA plates were prepared on which 10⁴ T. mentagrophytes cells were evenly spread. Next, discs from insoles and leather were treated as follows (with either agent or control vehicle): discs were pretreated with a single spray, spraying for 15 second or 30 second. Other discs were immersed in agent or vehicle for 30 min. Following this treatment, discs were air-dried by placing them in a Petri plate for 1 min. These dried discs were then placed (drug side down) on the seeded PDA plates. Plates were then incubated for 4 days at 30° C. Following incubation, fungal growth was recorded. Active agents showed a clearance zone around the biopsy disc (FIG. 1A, arrow), while inactive agents showed fungal growth all around the disc (FIG. 1B). Diameter of the clearance zone (CZD) was measured. The relative activity of different agents and control were assessed. In this assay, active agents had higher CZD than inactive or less active ones.

Post-treatment assays: To evaluate the ability of various agents to treat infected shoes, PDA plates were prepared on which 104 T. mentagrophytes cells were evenly spread on their surface. Next, untreated biopsy discs were placed on these PDA plates and incubated for 4 days at 30° C. Incubating the biopsy discs in this manner allowed the fungi to invade the discs. Infected discs were picked and post-treated with different agents by spraying. Post-treated discs were allowed to air dry and were then placed on fresh, uninoculated PDA plates and incubated for 4 additional days at 30° C. Incubation of the discs under these conditions allows any fungi that are not killed by the sprayed agent to grow. In other words, agents that are effective in the treatment of shoe material will not show any fungal growth around the disc biopsy (FIG. 1C). In contrast, discs treated with ineffective agents will show fungal growth emanating from them (FIG. 1D). Diameter of the growth zone (GZD) was determined as a measure of the activity of the agent tested. In this assay, inactive or less active agents had higher GZD than active agents, while active agents did not show any fungal growth (GZD=0).

### Scanning Electron Microscopy (SEM).

Scanning electron microscopy (SEM) was used to monitor the ability of agents to eradicate fungal growth on shoe insoles or leather biopsy discs. Pre- and post-treated discs were processed for SEM by the method of Chandra et al. One set of discs was used as a control in which no drug pre- or post-treatment was performed. In addition, one set of biopsy discs was used as blank where no fungal cells or drug were added. Following treatment, discs were fixed with 2% glutaraldehyde for 2 h, and then washed with sodium cacodylate buffer (three times for 10 minutes each). The discs were then treated with 1% osmium tetraoxide (for 1 h at 4° C.) followed by a series of washing with sodium cacodylate buffer, followed by a two times washing with distilled water. Next, the discs were treated with 1% tannic acid washed three times with distilled water, and followed by 1% uranyl acetate with two water washings. The samples were then dehydrated through a series of ethanol solutions (range from 25% (vol/vol) ethanol in distilled water to absolute ethanol). Prepared samples were then sputter coated with Au/Pd (60/40) and viewed with Amray 1000B scanning electron microscope.

### Results

Minimum Inhibitory Concentration (MIC) and Minimum Fungicidal Concentration (MFC):

Evaluation of the inhibitory activity of various agents showed that these agents were effective in inhibition of dermatophytes, yeasts and bacteria to varying degrees. Data from these MIC/MFC studies are summarized in Table 1 (for details of the MIC/MFC data, see Tables 2-5). Summary of the antifungal and antibacterial activity of different synthetic and natural products tested is summarized below.

Antimicrobial activity of Synthetic Agents:

Terbinafine: Our results showed that terbinafine was highly active against all isolates of the three dermatophytes genera tested where low MIC values were noted (MIC range=0.008-0.06 μg/mL). In addition, this agent was able to kill spores of these dermatophytes as demonstrated by low MFC values (MFC range was between 0.03-0.125 μg/mL). Evaluation of the anti-yeast activity of terbinafine showed that this agent possesses high activity against all C. parapsilosis isolates tested (MIC values for all isolates was 0.25 μg/mL). In this regard, C. parapsilosis is a known skin normal flora inhabitant. Our data showed that terbinafine was less active against C. albicans compared to C. parapsilosis with one to three fold higher MIC values against the majority of isolates tested relative to C. parapsilosis (MIC values for 5 strains ranged between 0.5 and −2 μg/mL). Interestingly, terbinafine exhibited no effect against one C. albicans strain (strain 8280 where the MIC was ≧64 μg/mL). In contrast to the high activity of terbinafine seen against dermatophytes and yeast strains, this agent did not show any antibacterial activity against all bacterial strains examined (MIC >64 μg/mL for all strains tested).

Tolnaftate: Evaluation of the antifungal activity of tolnaftate showed that this agent is highly active against the dermatophytes tested both in fungal inhibition (MIC range against all dermatophytes tested was 0.008-0.125 μg/mL) and spore killing (MFC range was 0.06-0.125 μg/mL). Tolnaftate inhibitory and sporicidal activity was similar to terbinafine or slightly (one dilution) higher. Evaluation of the anti-yeast activity of tolnaftate showed that this agent has a reduced activity against yeast compared to terbinafine. Elevated MICs for tolnaftate was observed against all C. albicans strains tested (MIC value for all strains was >64 μg/mL). While activity of tolnaftate against C. parapsilosis was strain-dependent with one strain (#7629) showing low MIC (0.5 μg/mL), while the other isolates exhibited relatively high MIC values (MIC=8-16 μg/mL). Susceptibility testing of bacteria to tolnaftate showed that this agent had no S. aureus antibacterial activity (MIC for all strains tested was >64 μg/mL), while possessing some strain-dependent activity against S. epidermidis strains: two strains had MIC values of 2 μg/mL, while the remaining four exhibited MICs ranging between 16 and >64 μg/mL.

Miconazole: Susceptibility testing of dermatophytes against miconazole showed that this agent possesses a potent antifungal activity against T. mentagrophytes, T. rubrum, and E. floccosum with MIC values ranging from 0.06 to 0.125 μg/mL. Compared to terbinafine and tolnaftate, miconazole had a slightly lower activity. Moreover, unlike these agents, miconazole was static against dermatophytes (MFC of miconazole against all T. mentagrophytes isolates and the majority of T. rubrum and E. floccosum isolates tested was ≧8 μg/mL). Our data show that miconazole possesses a modest anti-yeast activity. In general, the MIC values of miconazole against both C. albicans and C. parapsilosis were higher than those obtained for terbinafine. C. albicans showed some strain-dependent susceptibility against miconazole, with an MIC=1-2 μg/mL for four isolates, 16 μg/mL for another and >16 μg/mL for the remaining albicans strain (8280). MIC values of miconazole against C. parapsilosis were also strain dependent (MICs ranging from 4 to >16 μg/mL). In contrast to terbinafine and tolnaftate (which had no activity against bacteria), miconazole was active against both S. aureus and S. epidermidis isolates tested (MIC values against all Staphylococcus isolates were between 0.5 and 2 μg/mL).

Cedrus oil: Antifungal susceptibility testing of cedrus oil showed that this natural oil possessed acceptable antifungal activity against dermatophytes in vitro with MIC ranging between 0.5 and 2 μg/mL. In addition, cedrus oil exhibited species-dependent cidality: MFC against T. mentagrophytes was noticeably higher (MFC=4-16 μg/mL) than against E. floccosum, and T. rubrum isolates (MFC=0.5-4 μg/mL). Results are detailed in Table 1.

Tea Tree Oil: Antifungal susceptibility testing of dermatophytes against tea tree oil showed that this natural product is highly active in inhibiting and spore killing of these fungi (MIC range=0.125-0.4, while MFCs were =0.25 to >4 μg/mL against all dermatophytes tested). Moreover, the MIC and MFC values of tea tree oil against dermatophytes were lower than those noted for cedrus oil. A majority of the yeast isolates were resistant to tea tree oil (with an MIC>4 μg/mL). Interestingly, one C. albicans isolates (8280) was susceptible to tea tree oil, although the same isolate was resistant to terbinafine, tolnaftate, and miconazole (with an MIC of 64, >64, and >16 μg/mL, respectively). This finding is very interesting because it indicates that combining tea tree oil with any of the three agents may provide enhanced antifungal activity, suggesting that adding tea tree oil to any of the antifungals may provide a broad coverage against resistant isolates (MIC>4 μg/mL). The possibility of combining tea tree oil with different agents against this resistant fungus was evaluated (see below). The bacterial strains tested were not susceptible to tea tree oil. Results are detailed in Table 1.

Antimicrobial activity of all effective essential oils against dermatophytes known to grow on skin, hair, and nails, causing tinea pedis and tinea ungunium/onychomycosis, yeasts known to cause nail and cutaneous infections, and bacteria that can cause infection or generate unpleasant non-disease odor.

### Activity Against Dermatophytes:

In these studies we evaluated the activity of essential oils against dermatophytes, yeast and bacteria. Table 7 presents a summary of the anti-dermatophyte activity of essential oils. As can be seen, the five essential oils tested exhibited potent antifungal activity against dermatophytes with MICs ranging between 0.125 and 0.5 μg/mL.

### Activity Against Yeast:

Next we tested the ability of these oils to inhibit yeast (C. albicans and C. parapsilosis). As can be seen in Table 8, four of the essential natural oils (clover bud, lemongrass, spearmint, and tea tree oils) were active against these clinically important fungi, with MIC range between 0.063-0.5 μg/mL. The only exception was sandalwood oil, which had an MIC of 4 to >16 μg/mL (Table 3). These results suggested that sandalwood oil exhibited no inhibitory activity against Candida species and strains tested.

### Activity Against Bacteria:

We next tested the in vitro activity of the essential natural oils against: (a) odor-producing (Micrococcus and Corynebacteria) bacteria, and (b) Staphylococcus aureus (a major cause of cellulitis). As seen in Table 9, clove bud, lemongrass, and sandalwood oils were active against the odor-producing bacteria tested (MIC=0.25-2 μg/mL), while spearmint and tea tree oils did not show in vitro activity (MIC=2-8 μg/mL). Furthermore, clove bud, lemongrass, and sandalwood oils showed some activity (MIC=0.25-8 μg/mL) against Staphylococcus. Moreover, lemongrass tended to have one to two dilutions lower MIC than clove and sandalwood oils, indicating it is more active. In contrast, spearmint and tea tree oil did not show noticeable activity against any of the pathogenic bacterial isolates tested (MIC=8-32 μg/mL). These studies showed that clove bud and lemongrass had the broadest antimicrobial activity compared to the other essential oils and are viable candidates for use as natural products to prevent and treat tinea pedis, onychomycosis, and skin infections.

### Evaluation of the Activity of Combination of Antifungal Agents and Essential Oils Against Microorganisms Causing Superficial Fungal Infections, Including but not Limited to Tinea Pedis and Tinea Unguium/Onychomycosis.

To assess the potential for using antifungal synthetic agents (e.g. terbinafine, tolnaftate and miconazole) and essential oils (e.g. clove bud, lemongrass, sandalwood, spearmint and tea tree oil) in combination, we evaluated the ability of essential oils to inhibit terbinafine-resistant T. rubrum and C. albicans strain (strain number MRL 8280) that exhibits multi-resistance to terbinafine, miconazole and tolnaftate. As shown in Table 10, all the terbinafine-resistant T. rubrum isolates tested were susceptible to the essential natural oils, with an MIC range of 0.031 to 0.25 μg/mL. The most potent oil was lemongrass which showed very low MICs against these Trichophyton isolates.

Similarly, the essential oils were effective in inhibiting the multi-resistant C. albicans strain. The most effective essential oil in inhibiting this resistant strain was lemongrass (see Table 11).

### CONCLUSIONS

Based on these data it is concluded that: 1) essential oils (especially lemongrass and clove bud oils) can be used singly as natural products to inhibit microorganisms that infect the skin, hair and nails; and 2) combining essential oils with a synthetic or semisynthetic antifungal provides a broad spectrum activity, treats terbinafine resistant Trichophyton rubrum, multi-drug resistant Candida, and odor causing bacteria, as well as allows the use of lower concentrations of synthetic agents when combined with essential oils. Our method identified antimicrobial “systems” that have potent antifungal and antibacterial activity and provides an effective means for preventing and treating fungal infections.

Having shown that terbinafine, tolnaftate, and essential oils possess potent anti-dermatophyte activity against microorganisms that colonize and infect the foot using in vitro susceptibility assays, we next investigated the activity of these agents against dermatophytes using the shoe disc bioassay we developed, and SEM techniques.

Effect of Pretreatment of Shoe Insoles and Leather Surfaces with Synthetic and Natural Products on Preventing Dermatophyte Shoe Contamination

### Pretreatment of Insoles

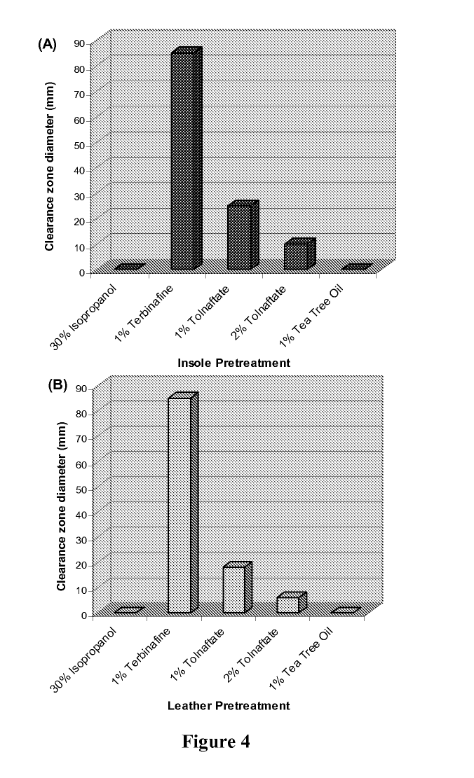

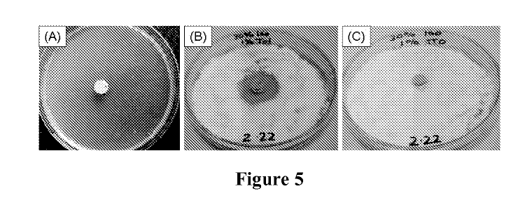

To determine the ability of terbinafine, tolnaftate, and tea tree oil to prevent shoe contamination we used the pretreatment insole biopsy bioassay method described above. As shown in FIG. 4 (and Table 11, representative images in FIG. 5), pretreatment of insoles with terbinafine 1% solution resulted in complete inhibition of fungal growth (CZD=85 mm, fungal inhibition reached to the edge of the Petri dish) compared to vehicle control (CZD=0 mm). This complete inhibition was observed even when the insoles discs were pretreated with a single spray. Pre-treatment of biopsy discs with tolnaftate (1% and 2%) also inhibited fungal growth (CZD=25 mm and 11 mm, respectively) compared to vehicle control, albeit to a lesser extent than terbinafine. Because tolnaftate dissolves better in acetone, we repeated some experiments using acetone as a vehicle. Our data showed that tolnaftate has a potent preventive activity against dermatophytes infecting shoes (FIG. 5). Increasing the concentration of tolnaftate to 3% and 4% increased the activity. In contrast, 1% tea tree oil had no inhibitory prevention effect (CZD=0 mm). Taken together, these data show that pretreatment of shoe insoles and leather material with terbinafine or tolnaftate is an effective way to prevent fungal contamination of shoes. Importantly, these agents were superior to the marketed Dr. Scholl's brand in preventing fungal contamination of insoles.

### Pretreatment of Leather

To determine whether pretreatment of leather biopsy discs with terbinafine, tolnaftate, or tea tree oil can prevent growth of T. mentagrophytes, we tested their activity using the bioassay method described above. As shown in FIG. 4B, pretreatment of leather disc with vehicle did not result in any inhibition (CZD=0 mm), while terbinafine pretreatment resulted in complete inhibition of fungal growth (CZD=85 mm, also see Table 6). Pretreatment of leather biopsies with 1% tolnaftate resulted in inhibition of fungal growth (CZD=16 mm, FIG. 4B). However, pretreatment of leather disc with 1% tea tree oil alone did not inhibit fungal growth (CZD=0 mm).

These data indicate that pre-treatment of leather material with terbinafine or tolnaftate is an effective way for preventing fungal contamination of the leather used in shoes.

Effect of Post-Contamination Treatment with Synthetic and Natural Products on Eradication of Pre-Established Dermatophyte Contamination on Insoles and Leather Surfaces

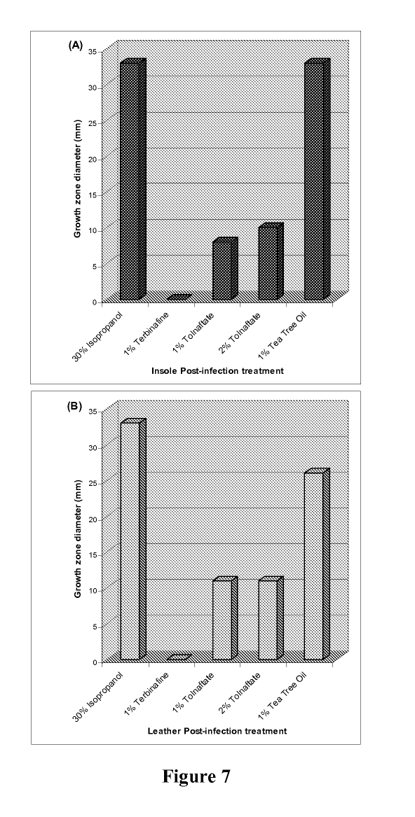

To determine the ability of terbinafine, tolnaftate, or tea tree oil to treat T. mentagrophytes contamination already established on shoe insoles, we determined the effect of post-treating infected insoles with these agents on their ability to clear the established fungal contamination using our post-treatment shoe biopsy disc assay developed (see above). As shown in FIG. 7A (and Table 6), while the vehicle control failed to treat already established fungal contamination as evidenced by the presence of fungal regrowth (GZD=33 mm of growth), terbinafine completely eradicated established contamination on insoles (GZD=0 mm). Tolnaftate (both 1% and 2%) were also effective in clearing the contamination of insole, although some minimal regrowth was observed (GZD=8 mm and 10 mm, respectively). In contrast, tea tree oil was not able to treat the contamination present on insole biopsies (GZD=33 mm).

### Post Contamination Treatment of Leather

Next, we determined whether terbinafine, tolnaftate, or tea tree oil can treat T. mentagrophytes contamination already established on leather biopsy discs. As shown in FIG. 7B (and Table 6), terbinafine completely cleared the established contamination on leather disc (GZD=0 mm). Moreover, tolnaftate (1% and 2%) also reduced contamination of leather (GZD=11 mm for both concentrations). Tea tree oil induced very minimal inhibition of fungal growth on the infected leather biopsies compared to vehicle control (GZD=26 mm versus 33 mm). These data clearly demonstrate that post treatment of insoles and leather with terbinafine and tolnaftate is an effective way for treating infected shoes.

### Effect of Combination of Tolnaftate and Tea Tree Oil on Preventing Dermatophyte Shoe Contamination

To determine whether using combination of synthetic compounds and essential oils will allow the use of low concentrations of synthetic drugs, we tested the ability of combination of 0.01% tolnaftate and 3% tea tree oil to prevent shoe contamination using the pre- and post-treatment insole bioassays described above.

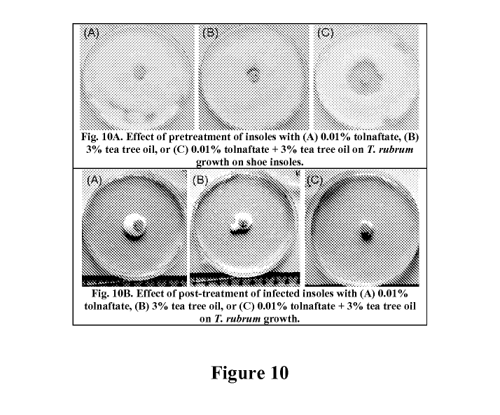

As shown in FIG. 10, pretreatment of insoles with 0.01% terbinafine or 3% tea tree oil singly did not result in any inhibition of fungal growth. In contrast, the combination of 0.01% terbinafine and 3% tea tree oil induced a noticeable inhibition of fungal growth (FIG. 10C).

Furthermore, while 0.01% tolnaftate or 3% tea tree oil did not prevent growth of fungus on insole after post-treatment (FIG. 10A (A)-(B), post-contamination treatment with the combination of these agents reduced fungal growth (FIG. 10A (C).

These data show that combining tolnaftate and tea tree oil will allow the use of low concentration of tolnaftate to prevent and treat shoe contamination.

### Scanning Electron Microscopy Analyses

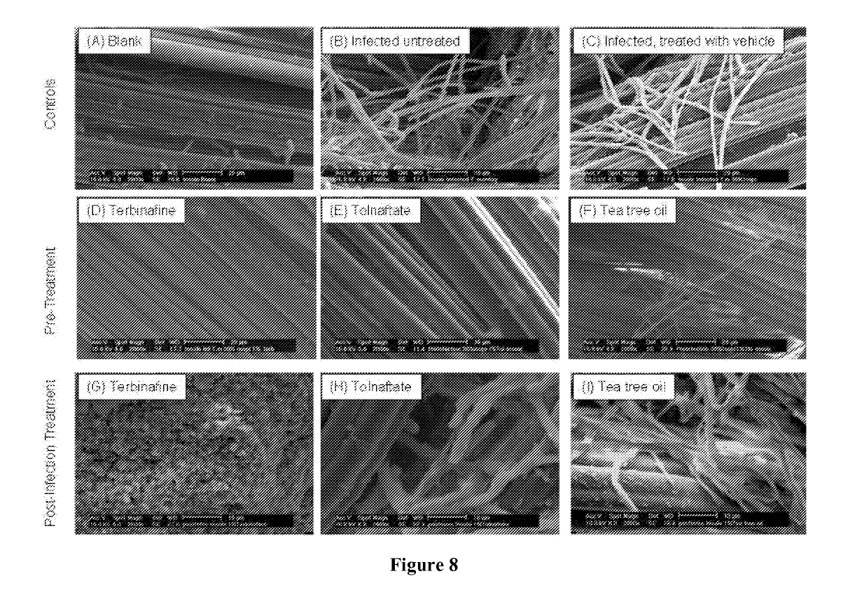

To determine the effect of synthetic and natural products (pre- and post-contamination treatments) on the ability of T mentagrophytes to grow on insole biopsies, we performed SEM analysis. As shown in FIG. 8, pretreatment of insole with the vehicle had no effect on fungal growth (FIG. 8C), while terbinafine and tolnaftate completely eradicated fungal growth (FIGS. 8D,E; no fungal elements were seen). However, and similar to the bioassay studies, pretreatment with tea tree oil reduced the fungal growth on insoles but did not eliminate it from the biopsy disc (FIG. 8F). Post-contamination treatment of insole with terbinafine resulted in complete clearance of fungal growth (FIG. 8G), while treatment with tolnaftate was minimally effective, as shown by the presence of several filaments (FIG. 8H). In contrast, post-contamination treatment with tea tree oil had no activity against T mentagrophytes (FIG. 8I).

These data show that pre- and post-treatment with terbinafine is highly effective in eradicating fungal elements from shoe material infected with T. mentagrophytes. Additionally, tolnaftate was effective in eradicating fungal elements only if insole biopsies were pretreated with this agent.

Taken together, these data indicate that pre- and post-treatment of insoles with either terbinafine or tolnaftate is effective in preventing the fungal colonization of, and treatment of already existing, fungal growth on insoles.

Our data also demonstrate that combining tolnaftate and tea tree oil will allow the use of low concentration of tolnaftate to prevent and treat shoe contamination

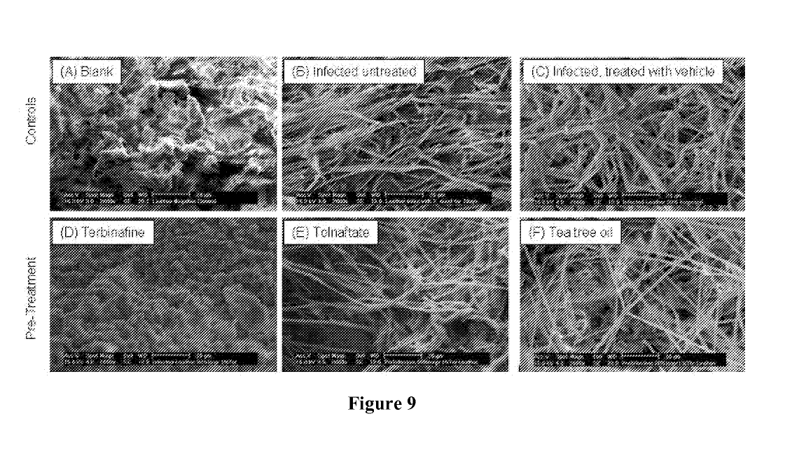

We also used SEM analyses to determine the effect of terbinafine, tolnaftate and tea tree oil pretreatment on their ability to prevent T. mentagrophytes growth on leather biopsies. As shown in FIG. 9, pretreatment of leather biopsy disc with terbinafine completely eradicated fungal growth, with no fungal elements seen (FIG. 9D), compared to untreated leather disc (FIG. 9B) or vehicle-treated disc (FIG. 9C), where massive fungal elements can be seen invading the leather material. Pretreatment with tolnaftate appeared to reduce the fungal density on leather discs, but did not completely eradicate dermatophyte growth (FIG. 9E). Discs pretreated with tea tree oil did not show any effect on fungal growth (FIG. 9F), and were similar in appearance to the discs pretreated with isopropanol, the vehicle control.

Taken together, these results revealed that pre-treatment of leather with terbinafine was highly effective in preventing and eradicating fungal elements from leather material infected with T. mentagrophytes, while tolnaftate was minimally effective. In contrast, tea tree oil was ineffective in eradicating contamination of leather discs.

In summary, our findings show that:

Among the synthetic agents tested (terbinafine, tolnaftate, miconazole):

The most active agent inhibiting dermatophytes was terbinafine, followed by tolnaftate and miconazole.

Terbinafine and tolnaftate were able to kill dermatophyte fungal spores that may infect hair, nails, and skin, while miconazole is static against dermatophyte spores.

Terbinafine and miconazole were also effective against the yeast Candida species, but in a strain- and species-dependent manner.

Miconazole exhibited activity against bacterial species, while tolnaftate exhibited strain-dependent inhibition of S. epidermidis.

Among the natural products tested were tea tree oil, cedrus oil, clove bud, lemongrass oil, sandalwood oil, and spearmint oil.

Essential oils have a broad antimicrobial activity covering dermatophytes, yeast and bacteria that infect the skin, hair and nails. The most active essential oil was lemongrass followed by clove bud.

Essential oils possess inhibitory activity against bacteria that produces unpleasant and unacceptable odors and Staphylococcus aureus (a major cause of cellulitis).

The essential natural oils have potent in vitro activity against terbinafine-resistant dermatophytes, as well as multi-resistant C. albicans. Lemongrass possesses the most potent activity in this regard.

In bioassay studies, terbinafine and tolnaftate pretreatment were able to inhibit fungi on insoles and leather shoe biopsies compared to vehicle control. Terbinafine was the most active pre-treatment agent.

Terbinafine post-treatment was able to treat established fungal contamination on shoe biopsy discs. Although tolnaftate showed antifungal activity as a post-treatment agent, its activity was less than that of terbinafine. Tea tree oil was ineffective as a post-treatment agent.

Combining a synthetic antifungal agent with an essential oil allows the use of low doses of the synthetic antifungal to prevent and treat infections of the skin, nails, and hair.

Our data indicate that combining agents is likely to provide benefit by expanding the spectrum of activity of an antifungal through the inhibition of resistant fungal strains.

In conclusion, the invented antimicrobial system has potent antifungal and antibacterial activity and provides an effective means for preventing and treating fungal infections of the skin, hair and nails.

In the following Tables, MIC₅₀ and MIC₉₀ are defined as the minimal concentrations of a compound that can inhibit 50% and 90% of the tested organisms, respectively.

TABLE 1
Range of MICs (μg/ml) and MFCs (μg/ml) of Terbinafine, Tolnaftate, Miconazole,
Tea Tree Oil and Cedrus Oil against Dermatophytes, Yeasts and Bacteria

| | Terbinafine | Tolnaftate | Miconazole | Tea tree oil | Cedrus oil |
|---|---|---|---|---|---|
| Organism | (μg/ml) | (μg/ml) | (μg/ml) | (μg/ml) | (μg/ml) |
| All | | | | | |
| Dermatophytes | | | | | |
| MIC Range | 0.015 | 0.125 | 0.015-0.25 | 0.0625-0.25 | 0.25-1.0 |
| MFC Range | 0.03-0.125 | 0.06-1.0 | 0.5->8.0 | 0.125->2 | 0.5-8 |
| All Yeasts | | | | | |
| MIC range | 0.25->64 | 0.5->64 | 1->16 | 0.125->2 | ND* |
| MFC range | — | — | 0.5-2 | — | ND* |
| All Bacteria | | | | | |
| MIC range | >64 | 2->64 | 0.5-2 | >4 | ND* |
| MFC range | — | — | — | — | ND* |

*ND—not determined

TABLE 2
Minimum Inhibitory Concentration (MIC, μg/mL) and Minimum Fungicidal Concentration
(MFC, μg/mL) of Terbinafine, Tolnaftate, and Miconazole Against Dermatophytes

| | Terbinafine | | Tolnaftate | | Miconazole | |
|---|---|---|---|---|---|---|
| Organism | MIC | MFC | MIC | MFC | MIC | MFC |
| E. floccosum | | | | | | |
| 1666 | 0.06 | 0.06 | 0.06 | 0.25 | 0.125 | 2 |
| 1798 | 0.03 | 0.03 | 0.06 | 0.25 | 0.125 | 0.5 |
| 1925 | 0.03 | 0.06 | 0.06 | 0.25 | 0.06 | 8 |
| 1926 | 0.06 | 0.06 | 0.125 | 0.5 | 0.125 | >8 |
| 1961 | 0.06 | 0.125 | 0.125 | 0.5 | 0.125 | >8 |
| 2165 | 0.06 | 0.125 | 0.06 | 0.25 | 0.125 | >8 |
| MIC Range (n = 6) | 0.03-0.06 | 0.03-0.125 | 0.06-0.125 | 0.25-0.5 | 0.06-0.125 | 0.5->8 |
| MIC₅₀ | 0.06 | 0.06 | 0.06 | 0.25 | 0.125 | 8 |
| MIC₉₀ | 0.06 | 0.125 | 0.125 | 0.5 | 0.125 | >8 |
| T. rubrum | | | | | | |
| 1967 | 0.015 | 0.125 | 0.015 | 0.06 | 0.125 | 8 |
| 2098 | 0.015 | 0.06 | 0.015 | 0.06 | 0.125 | 4 |
| 2246 | 0.008 | 0.06 | 0.008 | 0.06 | 0.125 | 1 |
| 8063 | 0.015 | 0.06 | 0.008 | 0.06 | 0.125 | 8 |
| 8071 | 0.015 | 0.06 | 0.015 | 0.06 | 0.25 | 8 |
| 8092 | 0.015 | 0.03 | 0.015 | 0.125 | 0.25 | 8 |
| MIC Range (n = 6) | 0.008-0.015 | 0.03-0.125 | 0.008-0.015 | 0.06-0.125 | 0.125-0.25 | 1-8 |
| MIC₅₀ | 0.015 | 0.06 | 0.015 | 0.06 | 0.125 | 8 |
| MIC₉₀ | 0.015 | 0.125 | 0.015 | 0.125 | 0.25 | 8 |
| T. mentagrophytes | | | | | | |
| 1720 | 0.004 | ND* | 0.008 | ND | 0.03 | ND |
| 2124 | 0.03 | 0.125 | 0.06 | 0.5 | 0.25 | >8 |
| 2125 | 0.03 | 0.125 | 0.06 | 0.5 | 0.25 | >8 |
| 2126 | 0.004 | ND | 0.004 | ND | 0.015 | ND |
| 2127 | 0.03 | 0.06 | 0.06 | 1 | 0.25 | >8 |
| 2128 | 0.03 | 0.125 | 0.125 | 1 | 0.125 | >8 |
| MIC Range (n = 6) | 0.004-0.03 | 0.06-0.125 | 0.008-0.125 | 0.5-1 | 0.015-0.25 | >8->8 |
| MIC₅₀ | 0.03 | 0.125 | 0.06 | 0.5 | 0.25 | >8 |
| MIC₉₀ | 0.03 | 0.125 | 0.125 | 1 | 0.125 | >8 |
| All | | | | | | |
| dermatophytes | | | | | | |
| MIC Range (n = 18) | 0.008-0.015 | 0.03-0.125 | 0.008-0.125 | 0.06-1 | 0.015-0.25 | 0.5->8 |
| MIC₅₀ | 0.03 | 0.06 | 0.06 | 0.125 | 0.125 | 8 |
| MIC₉₀ | 0.06 | 0.125 | 0.125 | 1 | 0.25 | >8 |

*ND = Not Determined

TABLE 3
Minimum Inhibitory Concentration (MIC, μg/mL) and
Minimum Fungicidal Concentration (MFC, μg/mL) of
Cedrus Oil and Tea Tree Oil against Dermatophytes

| | Cedrus Oil | | Tea Tree Oil | |
|---|---|---|---|---|
| Organism | MIC | MFC | MIC | MFC |
| E. floccosum | | | | |
| 1666 | 0.5 | 2 | 0.25 | 2 |
| 1798 | 0.25 | 1 | 0.25 | 1 |
| 1925 | 0.5 | 1 | 0.5 | 2 |
| 1926 | 0.5 | 1 | 0.5 | 2 |
| 1961 | 1 | 4 | 0.5 | 2 |
| 2165 | 0.5 | 4 | 0.5 | 1 |
| MIC Range (n = 6) | 0.25-1 | 1-4 | 0.25-0.5 | 1-2 |
| MIC₅₀ | 0.5 | 1 | 0.5 | 2 |
| MIC₉₀ | 1 | 4 | 0.5 | 2 |
| T. rubrum | | | | |
| 1967 | 0.5 | 2 | 0.25 | 2 |
| 2098 | 0.5 | 2 | 0.5 | 2 |
| 2246 | 0.5 | 0.5 | 0.5 | 1 |
| 8063 | 1 | 2 | 0.5 | 2 |
| 8071 | 1 | 4 | 0.5 | 4 |
| 8092 | 1 | 2 | 0.5 | 2 |
| MIC Range (n = 6) | 0.5-1 | 0.5-4 | 0.25-0.5 | 1-4 |
| MIC₅₀ | 0.5 | 2 | 0.5 | 2 |
| MIC₉₀ | 1 | 4 | 0.5 | 2 |
| T. mentagrophytes | | | | |
| 1720 | 0.25 | ND | 0.25 | >4 |
| 2124 | 2 | 8 | 0.125 | 4 |
| 2125 | 1 | 16 | 0.25 | 4 |
| 2126 | 0.25 | ND | 0.25 | >4 |
| 2127 | 0.5 | 8 | 0.25 | 4 |
| 2128 | 0.5 | 4 | 0.25 | 4 |
| MIC Range (n = 6) | 0.25-2 | 4-16 | 0.125-0.25 | 4->4 |
| MIC₅₀ | 0.5 | 8 | 0.25 | 4 |
| MIC₉₀ | 0.5 | 16 | 0.25 | >4 |
| All dermatophytes | | | | |
| MIC Range (n = 18) | 0.5-2 | 1-16 | 0.125-0.5 | 0.25->4 |
| MIC₅₀ | 0.5 | 2 | 0.25 | 2 |
| MIC₉₀ | 1 | 8 | 0.5 | 4 |

TABLE 4
Minimum Inhibitory Concentration (MIC, μg/mL) of Terbinafine,
Tolnaftate, Miconazole, and Tea Tree Oil against Candida species.

| | Terbinafine | Tolnaftate | Miconazole | Tea Tree Oil |
|---|---|---|---|---|
| Strain | MIC | MIC | MIC | MIC |
| C. albicans | | | | |
| 1740 | 1 | >64 | 1 | >4 |
| 2108 | 0.5 | 64 | 2 | >4 |
| 2153 | 0.5 | >64 | 1 | >4 |
| 8280 | >64 | >64 | >16 | 0.25 |
| 8283 | 0.5 | >64 | 16 | 0.5 |
| 8364 | 2 | >64 | 2 | >4 |
| MIC Range | 0.5->64 | 64->64 | 1->16 | 0.25->4 |
| (n = 6) | | | | |
| MIC₅₀ | 0.5 | >64 | 2 | >4 |
| MIC₉₀ | 2 | >64 | 16 | >4 |
| C. parapsilosis | | | | |
| 7629 | 0.25 | 0.5 | 4 | 0.25 |
| 7668 | 0.25 | 8 | 16 | >4 |
| 7672 | 0.25 | 8 | 8 | >4 |
| 7995 | 0.25 | 8 | 4 | >4 |
| 8148 | 0.25 | 8 | >16 | 2 |
| 8442 | 0.25 | 16 | 4 | >4 |
| MIC Range | 0.25-0.25 | 0.5-16 | 4->16 | 0.25->4 |
| (n = 6) | | | | |
| MIC₅₀ | 0.25 | 8 | 4 | >4 |
| MIC₉₀ | 0.25 | 8 | 16 | >4 |
| All yeasts | | | | |
| MIC Range | 0.25->64 | 0.5->64 | 1->16 | 0.25->4 |
| (n = 12) | | | | |
| MIC₅₀ | 0.25 | 16 | 4 | >4 |
| MIC₉₀ | 2 | >64 | >16 | >4 |

TABLE 5
Minimum Inhibitory Concentration (MIC, μg/mL) of Terbinafine, Tolnaftate,
Miconazole, and Tea Tree Oil against Staphylococcus species.

| | TERBINAFINE | TOLNAFTATE | MICONAZOLE | TEA TREE OIL |
|---|---|---|---|---|
| SPECIES | MIC | MIC | MIC | MIC |
| S. aureus | | | | |
| 93 | NON-VIABLE | NON-VIABLE | NON-VIABLE | NON-VIABLE |
| 730 | >64 | >64 | 2 | >4 |
| 732 | >64 | >64 | 2 | >4 |
| 733 | >64 | >64 | 2 | >4 |
| 734 | >64 | >64 | 2 | >4 |
| 8470 | >64 | >64 | 2 | >4 |
| MIC Range | >64 | >64 | 2 | >4 |
| (n = 6) | | | | |
| MIC₅₀ | >64 | >64 | 2 | >4 |
| MIC₉₀ | >64 | >64 | 2 | >4 |
| S. epidermidis | | | | |
| 8472 | >64 | 2 | 0.5 | >4 |
| 8473 | >64 | 16 | 1 | >4 |
| 8474 | >64 | 2 | 0.5 | >4 |
| 8475 | >64 | >64 | 1 | >4 |
| 8476 | >64 | >64 | 1 | >4 |
| 8477 | >64 | >64 | 1 | >4 |
| MIC Range | >64 | 2->64 | 0.5-1 | >4 |
| (n = 6) | | | | |
| MIC₅₀ | >64 | 16 | 1 | >4 |
| MIC₉₀ | >64 | >64 | 1 | >4 |
| All bacteria | | | | |
| MIC Range | >64 | 2->64 | 0.5-2 | >4 |
| (n = 12) | | | | |
| MIC₅₀ | >64 | >64 | 1 | >4 |
| MIC₉₀ | >64 | >64 | 2 | >4 |

TABLE 6
Effect of pretreatment and post-contamination treatment of leather and insole
biopsy discs with different agents on growth of T. mentagrophytes.

| | | Insoles Post- | | Leather Post- |
|---|---|---|---|---|
| | Insoles | contamination | Leather | contamination |
| | Pretreatment | Treatment | Pretreatment | Treatment |
| Spray | (CZD*, mm) | (GZD*, mm) | (CZD, mm) | (GZD, mm) |
| 30% Isopropanol | 0 | 33 | 0 | 33 |
| 1% Terbinafine | 85 | 0 | 85 | 0 |
| 1% Tolnaftate | 25 | 8 | 18 | 11 |
| 2% Tolnaftate | 10 | 10 | 6 | 11 |
| 1% Tea Tree Oil | 0 | 33 | 0 | 26 |
| 1% Tol 1% TTO** | 11 | 17 | 11 | 20 |
| 2% Tol 1% TTO | 19 | 8 | 15 | 11 |
| 2% Tol 2% TTO | 10 | 34 | 0 | 30 |
| 3% Tol 1% TTO | 25 | 11 | 20 | 11 |

*CZD—clearance zone diameter; GZD—growth zone diameter.
**Tol—Tolnaftate: TTO—tea tree oil.

TABLE 7
Activity of essential oils against dermatophytes

| | | Clove | Lemongrass | Sandalwood | Spearmint | Tea |
|---|---|---|---|---|---|---|
| Genus | Species | Bud Oil | Oil | Oil | Oil | Tree Oil |
| Epidermophyton | floccosum | 0.125 | 0.25 | 0.5 | 0.5 | 0.5 |
| Epidermophyton | floccosum | 0.125 | 0.25 | 0.25 | 0.125 | 0.5 |
| Trichophyton | mentagrophytes | 0.125 | 0.5 | 0.25 | 0.25 | 0.25 |
| Trichophyton | mentagrophytes | 0.125 | 0.25 | 0.25 | 0.25 | 0.25 |
| Trichophyton | rubrum | 0.125 | 0.25 | 0.5 | 0.25 | 0.5 |
| Trichophyton | rubrum | 0.125 | 0.25 | 0.5 | 0.5 | 0.5 |
| | MIC Range | 0.125-0.125 | 0.25-0.5 | 0.25-0.5 | 0.125-0.5 | 0.25-0.5 |
| | MIC₅₀ | 0.125 | 0.25 | 0.25 | 0.25 | 0.5 |
| | MIC₉₀ | 0.125 | 0.5 | 0.5 | 0.5 | 0.5 |

TABLE 8
Activity of essential natural oils against yeast Isolates

| | | Clove Bud | Lemongrass | Sandalwood | Spearmint | Tea |
|---|---|---|---|---|---|---|
| Genus | Species | Oil | Oil | Oil | Oil | Tree Oil |
| Candida | albicans | 0.125 | 0.063 | >16 | 0.5 | 0.25 |
| Candida | albicans | 0.125 | 0.25 | >16 | 2 | 1 |
| Candida | albicans | 0.5 | 0.125 | >16 | 2 | 1 |
| Candida | parapsilosis | 0.125 | 0.125 | 4 | 0.5 | 0.25 |
| Candida | parapsilosis | 0.125 | 0.125 | >16 | 0.5 | 0.25 |
| Candida | parapsilosis | 0.25 | 0.125 | >=16 | 1 | 0.25 |
| | MIC Range | 0.125-0.5 | 0.063-0.25 | 4->16 | 0.5-2 | 0.25-1 |
| | MIC₅₀ | 0.125 | 0.125 | >16 | 0.5 | 0.25 |
| | MIC₉₀ | 0.5 | 0.25 | >16 | 2 | 1 |

TABLE 9
Activity of natural oils against (A) odor-causing and (B) pathogenic bacterial isolates

| | | | | | | |
|---|---|---|---|---|---|---|
| | | Clove Bud | Lemongrass | Sandalwood | Spearmint | Tea Tree |
| MRL | | Oil MIC | Oil MIC | Oil MIC | Oil MIC | Oil |
| Number | Organism | (mg/mL) | (mg/mL) | (mg/mL) | (mg/mL) | (mg/mL) |

(A) Odor causing bacteria

| | | | | | | |
|---|---|---|---|---|---|---|
| 781 | Corynebacterium sp. | 2 | 0.25 | 0.5 | 8 | 8 |
| 782 | Corynebacterium sp. | 1 | 0.25 | 0.25 | 4 | 2 |
| 783 | Micrococcus luteus | 0.5 | 0.5 | 0.25 | 4 | 2 |
| 784 | Micrococcus luteus | 0.5 | 0.25 | 0.5 | 2 | 4 |
| | MIC Range (n = 4) | 0.5-2 | 0.25-0.5 | 0.25-0.5 | 2-8 | 2-8 |
| | MIC₅₀ | 0.5 | 0.25 | 0.25 | 4 | 2 |
| | MIC₉₀ | 2 | 0.5 | 0.5 | 8 | 8 |

(B) Pathogenic bacteria

| | | | | | | |
|---|---|---|---|---|---|---|
| 730 | S. aureus | 2 | 1 | 2 | 8 | 32 |
| 732 | S. aureus | 1 | 1 | 0.25 | 8 | NG |
| 733 | S. aureus | 1 | 0.5 | 0.5 | 8 | 8 |
| 8470 | S. aureus | 2 | 1 | 2 | 16 | 32 |
| | MIC Range (n = 4) | 1-2 | 0.5-1 | 0.25-2 | 8-16 | 8-32 |
| | MIC₅₀ | 2 | 1 | .25 | 8 | 32 |
| | MIC₉₀ | 2 | 1 | >2 | 8 | >32 |

TABLE 10
Activity of natural oils against terbinafine-resistant T. rubrum Isolates

| | MRL | Terbinafine | Clove Bud | Lemongrass | Sandalwood | Spearmint | Tea Tree |
|---|---|---|---|---|---|---|---|
| Organism | Number | MIC | oil MIC | oil MIC | oil MIC | oil MIC | oil MIC |
| T. rubrum | 666 | 16 | 0.25 | 0.063 | 2 | 2 | 4 |
| T. rubrum | 670 | 16 | 0.125 | 0.063 | 0.5 | 0.5 | 1 |
| T. rubrum | 671 | 4 | 0.125 | 0.063 | 1 | 0.5 | 1 |
| T. rubrum | 1386 | 4 | 0.125 | <=0.031 | 0.5 | 0.25 | 4 |
| T. rubrum | 1806 | 4 | 0.125 | <=0.031 | 0.5 | 0.25 | 0.5 |
| T. rubrum | 1807 | 4 | 0.125 | 0.063 | 0.5 | 0.5 | 4 |
| T. rubrum | 1808 | 16 | 0.125 | 0.25 | 0.5 | 0.5 | 2 |
| T. rubrum | 1809 | 16 | 0.25 | 0.125 | 2 | 2 | 4 |
| T. rubrum | 1810 | 4 | 0.063 | <=0.031 | 0.125 | 0.125 | 2 |
| T. rubrum | 2499 | 4 | 0.125 | 0.125 | 1 | 0.5 | 4 |
| T. rubrum | 2727 | 2 | 0.125 | <=0.031 | 0.25 | 0.125 | 1 |
| | MIC Range (n = 11) | 2-16 | 0.063-0.25 | <=0.031-0.25 | 0.125-1 | 0.125-2 | 0.5-4 |
| | MIC₅₀ | 4 | 0.125 | 0.063 | 0.5 | 0.5 | 2 |
| | MIC₉₀ | 16 | 0.25 | 0.125 | 2 | 2 | 4 |

TABLE 11
Activity of essential oils against a multi-resistant
strain of C. albicans (strain 8280).

| Essential Oil | MIC (μg/mL) |
|---|---|
| Clove Bud Oil | 0.125 |
| Lemongrass Oil | 0.063 |
| Sandalwood Oil | >16 |
| Spearmint Oil | 0.5 |

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

In view of the above, it will be seen that the several advantages of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method of treating a fungal infection on an animal epidermis, nail or hair, or in an orifice of an animal, the method comprising contacting the fungus infection with a composition comprising an antifungal botanical.

2. The method of claim 1, wherein the fungal infection is in an orifice.

3. The method of claim 2, wherein the orifice is a mouth or nose.

4. The method of claim 2, wherein the orifice is a vagina.

5. The method of claim 1, wherein the fungal infection is on an epidermis.

6. The method of claim 1, wherein the fungal infection is on a nail.

7. The method of claim 1, wherein the fungal infection causes or aggravates tinea corporis, tinea pedis, tinea cruris, tinea unguium/onychomycosis, tinea capitis, dandruff, or diaper rash.

8. The method of claim 1, wherein the fungal infection is of a Malassezia furfur, a Epidermophyton floccosum, a Trichophyton, a Dermatophilus congolensis, a Microsporum, a Malassezia ovale, an Aspergillus, a Blastomyces, a Candida, a Coccidioides, a Cryptococcus, a Histoplasma, a Paracoccidioides, a Sporothrix, a Zygomycetes, a Pseudallescheria, a Scedosporum or a Scopulariopsis.

9. The method of claim 1, wherein the antifungal botanical is an essential oil.

10. The method of claim 9, wherein the essential oil is clove bud oil, lemongrass oil, sandalwood oil, spearmint oil, carcacrol, thymol, a cardamom extract, caraway oil, a coriander extract, linalool, almond oil, or tea tree oil.

11. The method of claim 9, wherein the essential oil is clove bud oil or lemongrass oil.

12. The method of claim 9, wherein the essential oil comprises eugenol, terpinen-4-ol, cineole, cuminaldehyde, cinnamic acid, or perillaldehyde.

13. The method of claim 1, wherein the composition further comprises a second antifungal compound.

14. The method of claim 13, wherein the second antifungal compound is a synthetic or semisynthetic antifungal compound.

15. The method of claim 14, wherein the second antifungal compound is a synthetic antifungal compound.

16. The method of claim 15, wherein the synthetic antifungal compound is miconazole, terbinafine, tolnaftate or ciclopirox.

17. The method of claim 14, wherein the second antifungal compound is a semisynthetic antifungal compound.

18. The method of claim 17, wherein the semisynthetic antifungal compound is an echinocandin.

19. The method of claim 13, wherein the second antifungal compound is a botanical.

20. The method of claim 19, wherein the botanical is an essential oil.

21. The method of claim 1, wherein the animal is a human.

22. The method of claim 1, wherein the animal is a nonhuman vertebrate.

23. The method of claim 22, wherein the nonhuman vertebrate is a bird.

24. The method of claim 22, wherein the nonhuman vertebrate is a mammal.

25. The method of claim 22, wherein the nonhuman vertebrate is a farm animal.

26. The method of claim 24, wherein the farm animal is a cow, a pig, a chicken, or a horse.

27. The method of claim 22, wherein the nonhuman vertebrate is a companion animal.

28. The method of claim 27, wherein the companion animal is a dog, a cat, a hamster, a gerbil, a guinea pig, a mouse, a rat, a potbellied pig, a ferret, or a caged bird.

29. The method of claim 1, wherein the composition is applied directly on the fungal infected tissue.

30. The method of claim 1, wherein the composition is applied to an article that comes in contact with the fungus infection.

31. The method of claim 30, wherein the article is clothing, a towel, a comb, a brush, a diaper, human bedding, a shoe, or animal bedding.

32. The method of claim 1, wherein the composition is in the form of a cream, ointment, gel, liquid, solution, foam, powder, paste, gum, lacquer, shampoo, suspension, fog, spray, aerosol, pump spray, wipe or sponge.

33. The method of claim 1, further comprising contacting the fungal infection with a second composition, wherein the second composition comprises a separate antifungal compound.

34. The method of claim 33, wherein the separate antifungal compound is a synthetic or semisynthetic antifungal compound.

35. The method of claim 34, wherein the separate antifungal compound is a synthetic antifungal compound.

36. The method of claim 35, wherein the synthetic antifungal compound is miconazole, terbinafine, tolnaftate or ciclopirox.

37. The method of claim 34, wherein the separate antifungal compound is a semisynthetic antifungal compound.

38. The method of claim 35, wherein the semisynthetic antifungal compound is an echinocandin.

39. The method of claim 33, wherein the separate antifungal compound is a botanical.

40. The method of claim 39, wherein the botanical is an essential oil.

41. The method of claim 40, wherein the essential oil is clove bud oil or lemongrass oil.
